SEQUENCE LISTING (13 sequences)

SEQ ID NO: 1 - DNA, 24 bp, Artificial Sequence
  Oligonucleotide primer
cgttcaggcgcaacaggccgttgt

SEQ ID NO: 2 - DNA, 22 bp, Artificial Sequence
  Oligonucleotide primer
ggctgcgattggcgaggtagta

SEQ ID NO: 3 - DNA, 35 bp, Artificial Sequence
  Oligonucleotide primer
ggtgatcccgggcgtcaaacgtcagggcttcttta

SEQ ID NO: 4 - DNA, 35 bp, Artificial Sequence
  Oligonucleotide primer
ttgacgcccgggatcaccaaagagtccccgaacta

SEQ ID NO: 5 - DNA, 20 bp, Artificial Sequence
  Oligonucleotide primer
gcaacaactcctgctggtta

SEQ ID NO: 6 - DNA, 20 bp, Artificial Sequence
  Oligonucleotide primer
agaccgaggatcactttatc

SEQ ID NO: 7 - DNA, 19 bp, Artificial Sequence
  Oligonucleotide primer
aggaagtttgagaggataa

SEQ ID NO: 8 - DNA, 20 bp, Artificial Sequence
  Oligonucleotide primer
gtgtaggctggagctgcttc

SEQ ID NO: 9 - DNA, 20 bp, Artificial Sequence
  Oligonucleotide primer
catatgaatatcctccttag

SEQ ID NO: 10 - DNA, 2903 bp, Salmonella enteritidis
  misc_feature (0)...(0): Contig 1294 of the S. Enteritidis genome
ctgatcgaacaagccttcggcctgcagtttggcttttagctgctcatatttctgctgcaacaagccttcgcccgcgggctgcatactttcggcgatgatttgataatcgccgcgcggctcgtacagcgtaatgttggcgcgtaccagcacctgctgcccgtgctgcgggcggaacgtcacccggcgattgctgttacggaacatcgcacagcgcacctgagcggtatcgtctttgagcgtaaagtaccagtggcccgacgcaggctgcgtgaaattagaaatctcgccgctgatccatacctgtcccatctcctgttctaacagcagacgaaccgtctggttaaggcggcttacggtaaaaattgaggaagtttgagaggataacatgtgagcgggatcaaattctaaatcagcaggttattcaatcgatagtaacctgctcacgggggatcgcaagcactatttgcaaaaaaatgtagatgcaaccgattacgttctgtataatgccgcgggaatatttattaacctcccaggcgagatattgcccatgctacgtatcgctaaagaagctctgacgtttgacgacgtcctccttgttcccgctcactccaccgttttgccgaatactgccgatctcagcacgcagttgacgaaaactattcgtctgaatattcctatgctttctgcggcgatggacaccgtgacggaagcgcgcctggcaattgccctggcccaggaaggcggcattggttttatccacaaaaacatgtctattgagcgccaggcggaagaagttcgccgcgtgaagaaacacgagtccggcgtagtgaccgacccgcagaccgtcctgccaaccaccacgttgcatgaagtgaaagccctgaccgagcgtaacggttttgcgggctatccggtggtgactgaagataacgagctggtgggtatcatcaccggtcgtgacgtgcgttttgtgactgacctgaaccagccggtgagtgtctacatgacaccgaaagagcgtctggtgaccgttcgtgaaggcgaagcccgtgaagtcgtgctggcaaaaatgcacgaaaaacgcgtagaaaaagcgctggtcgttgatgataacttccatctgcttggcatgattaccgtaaaagatttccagaaagcggaacgtaaaccaaactcctgtaaagatgagcagggccgtttacgtgtcggcgcggcggtcggcgcaggcgcgggcaacgaagagcgcgttgacgcgctggtggcggcaggcgttgacgtactgctgatcgactcctctcacggtcactctgaaggcgtgttgcaacgtatccgtgagacgcgtgctaaatatcctgacctgcaaatcatcggcgggaacgttgcgacgggcgcaggcgctcgcgcactggcggaagccggttgcaacgcggtgaaagtgggtatcggcccgggttccatctgtacgactcgtatcgggactggtgtgggcgttccgcagatcaccgctgtttctgacgcggtggaagcgctggaaggcaccgggattccggttatcgctgacggcggtatccgtttctccgnnnacatcgccaaagccatcgccgcaggcgcgagcgcggtaatggtgggttctatgctggccggtaccgaagaatcnccgggcgaaatcgaactctaccagggccgttcttacaaatcttatcgcggtatgggttctctgggcgcgatgtccaaaggttcctccgaccgttacttccagagcgacaacgccgccgacaaactggtgccggaaggtatcgaaggccgcgtagcctataaaggtcgcctgaaagagatcattcaccagcagatgggcggcctgcgctcctgtatggggctgaccggttgtgctaccatcgacgaactgcgtactaaagcggagtttgtgcgtatcagcggtgcgggtatccaggaaagccacgttcacgacgtgaccatcaccaaagagtccccgaactaccgtctgggctcctgattttcttcgcccgaccttcgcgtcgggcgatttatttaatctgtttcacttgcctcggaataagcgtcaatgacggaaaacattcataagcatcgcatcctcattctggacttcggttctcagtacactcaactggttgcgcgccgcgtgcgtgagctgggtgtttactgtgaactgtgggcgtgggatgtgacagaagcacaaattcgtgacttcaacccaagcggcattattctttccggcggcccggaaagcaccaccgaagaaaacagcccgcgcgcgccgcagtatgtctttgaagcaggcgtgccggtatttggcgtttgctatggtatgcagaccatggcgatgcagcttggcggtcatgtagaaggttctaatgagcgtgaatttggttatgcgcaggtcgaagtgttgaccgacagcgcgctggttcgcggtattgaagattccctgaccgcagacggcaaaccgctgctggacgtgtggatgagccacggcgataaagtgacggcgattccgtccgacttcgtgaccgtcgccagcaccgagagctgcccgttcgccattatggccaacgaagaaaaacgcttctacggcgtacagttccacccggaagtgactcacacccgccagggtatgcgcatgctggagcgttttgtacgcgatatctgccagtgtgaagcgttgtggacgccggcgaagatcatcgatgacgccgtggcgcgcattcgcgagcaggtaggcgacgataaagtgatcctcggtctctccggcggcgtggattcttccgtcaccgccatgctgctgcaccgcgcgatcggtaaaaatctgacctgtgttttcgtcgacaacggcctgttgcgcctgaacgaagccgagcaggtga

SEQ ID NO: 11 - DNA, 1995 bp, Artificial Sequence
  Sequence of the delta-guaB fragment of S. Enteritidis cloned in pUC18
ggctgcgattggcgaggtagtagtccatcgccatgcccagacgctgctggcgganctggatctgacgcaacaactcctgctggttacggctgacaatttctgccgctgctgatggcgtagcgcgcgcaggtcggcgacaaaatcagctatcgtgacgtccgtttcgtgaccgacggcgctgaccaccggaatgcggctggcaaaaatcgcccgcgccacgcgttcgtcgttaaaactccacaaatcttccagcgaaccgccgccgcgcccgacgatcagtacatcacattcgccgcgcgcgttcgccagttcgatagcacgaacgatctgccccggcgcgtcgtcgccctggactgcggttggatagataataacgggcagggatgggtcacgacgctttagcacgtggagaatatcgtgcagcgccgcgccggttttcgaagtgatcaccccaacgcagtgggccggggagggcaacggctgtttatgctgctgatcgaacaagccttcggcctgcagtttggcttttagctgctcatatttctgctgcaacaagccttcgcccgcgggctgcatactttcggcgatgatttgataatcgccgcgcggctcgtacagcgtaatgttggcgcgtaccagcacctgctgcccgtgctgcgggcggaacgtcacccggcgattgctgttacggaacatcgcacagcgcacctgagcggtatcgtctttgagcgtaaagtaccagtggcccgacgcaggctgcgtgaaattagaaatctcgccgctgatccatacctgtcccatctcctgttctaacagcagacgaaccgtctggttaaggcggcttacggtaaaaattgaggaagtttgagaggataacatgtgagcgggatcaaattctaaatcagcaggttattcaatcgatagtaacctgctcacgggggatcgcaagcactatttgcaaaaaaatgtagatgcaaccgattacgttctgtataatgccgcggcaatatttattaacctcccaggtgagatattgcccatgctacgtatcgctaaagaagctctgacgtttgacgcccgggatcaccaaagagtccccgaactaccgtctgggctcctgattttcttcgcccgaccttcgcgtcgggcgatttatttaatctgtttcacttgcctcggaataagcgtcaatgacggaaaacattcataagcatcgcatcctcattctggacttcggttctcagtacactcaactggttgcgcgccgcgtgcgtgagctgggtgtttactgtgaactgtgggcgtgggatgtgacagaagcacaaattcgtgacttcaacccaagcggcattattctttccggcggcccggaaagcaccaccgaagaaaacagcccgcgcgcgccgcagtatgtctttgaagcaggcgtgccggtatttggcgtttgctatggtatgcagaccatggcgatgcagcttggcggtcatgtagaaggttctaatgagcgtgaatttggttatgcgcaggtcgaagtgttgaccgacagcgcgctggttcgcggtattgaagattccctgaccgcagacggcaaaccgctgctggacgtgtggatgagccacggcgataaagtgacggcgattccgtccgacttcgtgaccgtcgccagcaccgagagctgcccgttcgccattatggccaacgaagaaaaacgcttctacggcgtacagttccacccggaagtgactcacacccgccagggtatgcgcatgctggagcgttttgtacgcgatatctgccagtgtgaagcgttgtggacgccggcgaagatcatcgatgacgccgtggcgcgcattcgcgagcaggtaggcgacgataaagtgatcctcggtctctccggcggcgtggattcttccgtcaccgccatgctgctgcaccgcgcgatcggtaaaaatctgacctgtgttttcgtcgacaacggcctgttgcgcctgaacg

SEQ ID NO: 12 - DNA, 349 bp, Artificial Sequence
  Nucleotide sequence of the S. Enteritidis PCR fragment, which includes the guaB deletion, obtained using primer GuaB10
catgtgtgagcgggatcaaattctaaatcagcaggttattcaatcgatagtaacctgctcacgggggatcgcaagcactatttgcaaaaaaaatgtagatgcaaccgattacgttctgtataatgcccggcaatatttattaacctcccaggtgagatattcgccatgtacgtatcgctaaagaagccctgacgtttgacgcccgtgtaggctggagctgcttcgaagttcctatactttctagagaataggaactccggaataggaactaaggaggatattcatatgggatcaccaaagatccccgaactaccgtctgggctctgattttcttcgcccgaccttcgcg

SEQ ID NO: 13 - DNA, 1553 bp, Salmonella  Typhimurium
  misc_feature (0)...(0): guaB gene of S. Typhimurium LT2, section 117 of 220 of the complete genome
atttgcaaaaaaatgtagatgcaaccgattacgttctgtataatgccgcggcaatatttattaacctcccaggtgagatattgcccatgctacgtatcgctaaagaagccctgacgtttgacgacgtcctccttgttcccgctcactccaccgttttgccgaatactgccgatctcagcacgcagttgacgaaaactattcgtctgaatattcctatgctttctgcggcgatggacaccgtgacggaagcgcgcctggcaattgccctggcccaggaaggcggcattggttttatccacaaaaacatgtccattgagcgccaggcggaagaagttcgccgcgtgaagaaacacgagtccggcgtagtgaccgacccgcagaccgtcctgccaaccaccacgttgcatgaagtgaaagccctgaccgagcgtaacggttttgcgggctatccggtggtgactgaagataacgagctggtgggtatcatcaccggtcgtgacgtgcgttttgtgactgacctgaaccagccggttagcgtttacatgacgccgaaagagcgtctggtgaccgttcgtgaaggcgaagcccgtgaagtcgtgctggcaaaaatgcacgaaaaacgcgtagaaaaagcgctggtcgttgatgataacttccatctgcttggcatgattaccgtaaaagatttccagaaagcggaacgtaaaccaaactcctgcaaagatgagcagggccgtttacgtgtcggcgcggcggtcggcgcaggcgcgggcaacgaagagcgcgttgacgcgctggtggcggcaggcgttgacgtactgctgatcgactcctctcacggtcattcagaaggcgtgttgcaacgtatccgtgaaacccgtgctaaatatcctgacctgcaaatcatcggcggcaacgtcgcgacaggcgcaggcgctcgcgcactggcggaagccggttgcagcgcggtgaaagtcggtattggcccgggttccatctgtaccactcgtatcgtgactggcgtgggcgttccgcagatcaccgctgtttctgacgcagttgaagcgctggaaggtaccggtattccggttatcgctgacggcggtatccgtttctccggcgacatagccaaagcgattgccgcaggtgcaagcgcggtaatggtgggttccatgctggcgggtacggaagaatccccgggcgaaatcgaactctaccagggccgttcttacaaatcttaccgcggcatgggctcgctgggtgcgatgtccaaaggttcctccgaccgttacttccagagcgacaacgccgctgacaaactggtgccggaaggtatcgaaggtcgcgtagcctataaaggtcgcctgaaagagatcattcaccagcagatgggcggcctgcgctcctgtatggggctgaccggttgtgctaccatcgacgaactgcgtactaaagcggagtttgtgcgtatcagcggtgcgggcattcaggaaagccacgttcacgacgtgaccatcaccaaagagtccccgaactaccgtctgggctcctga

(19) United States
(12) Patent Application Publication
(10) Pub. No.: US 2010/0260794 A1
(43) Pub. Date: Oct. 14, 2010

(54) LIVE ATTENUATED SALMONELLA VACCINE

(75) Inventors: Henri Marcel Jozef De Greve, Sint-gillis (BE); Connie Theresia Adriaensen, Ghent (BE); Jean-Pierre Ernest Clement Hernalsteens, Sint-Pieters-Woluwe (BE)

Correspondence Address: KNOBBE MARTENS OLSON & BEAR LLP, 2040 MAIN STREET, FOURTEENTH FLOOR, IRVINE, CA, 92614, US

(73) Assignee: Vrije Universiteit Brussel

(21) Appl. No.: 12/293,785

(22) PCT Filed: Mar. 20, 2006
(86) PCT No.: PCT/BE06/00019

§ 371 Date: Sep. 24, 2009

(30) Foreign Application Priority Data
Mar. 20, 2006 (JP) 2006-077818

(51) Int. Cl. A61K 39/112; A61K 39/02; A61P 31/04

(52) U.S. Cl. 424/200.1; 424/258.1; 424/257.1

(57) ABSTRACT

The present invention relates to attenuated guaB deletion mutants of a bacterium infecting veterinary species, more in particular Salmonella enterica, to their use and production. The present invention further relates to live attenuated vaccines based on such mutants for preventing bacterial infections, and more in particular Salmonellosis, in a veterinary species, more in particular poultry.

### FIELD OF THE INVENTION

The present invention relates to attenuated bacterial mutants, in particular attenuated Salmonella enterica mutants, and to a live attenuated vaccine comprising same.

### STATE OF THE ART

Salmonellae are Gram-negative, facultative anaerobic, motile, non-lactose fermenting rods belonging to the family Enterobacteriaceae. Salmonellae are usually transmitted to humans by the consumption of contaminated foods and cause Salmonellosis.

Salmonellae have been isolated from many animal species including, cows, chickens, turkeys, sheep, pigs, dogs, cats, horses, donkeys, seals, lizards and snakes.

95% of the important Salmonella pathogens belong to Salmonella enterica, with S. enterica serovar Typhimurium (S. Typhimurium) and S. enterica serovar Enteritidis (S. Enteritidis) as the most common serovars.

Salmonella infections are a serious medical and veterinary problem world-wide and cause concern in the food industry. Contaminated food can not be readily identified.

Control of Salmonellosis is important, to avoid potentially lethal human infections and considerable economic losses for the animal husbandry industry.

The ubiquitous presence of Salmonella in nature complicates the control of the disease just by detection and eradication of infected animals.

Several control strategies based on the principles of competitive exclusion and vaccination have been tested to control the infection of e.g. poultry.

Vaccination of farm animals is often considered as the most effective way to prevent zoonoses caused by Salmonella.

Whole-cell killed vaccines and subunit vaccines are used in the prevention of Salmonella infections in animals and in humans with variable results. Inactivated vaccines in general provide poor protection against Salmonellosis.

Live attenuated Salmonella vaccines are potentially superior to inactivated preparations owing to: (i) their ability to induce cell-mediated immunity in addition to antibody responses; (ii) oral delivery with no risk of needle contamination; (iii) effectiveness after single-dose administration; (iv) induction of immune responses at multiple mucosal sites; (v) low production cost; and (vi) their possible use as carriers for the delivery of recombinant antigens to the immune system.

Few live attenuated Salmonella vaccines are actually on the market because results with attenuated mutant strains have not always been good.

For example, poultry has been vaccinated with aro- or cAMP mutants; yet many chicks died shortly after vaccination. As the infection by Salmonella often occurs very early, vaccination of very young chicks is crucial. However, at this age these are very sensitive to Salmonella, possibly due to the immaturity of their immune system. In addition to poor protection of the vaccinated chicks, a prolonged excretion of the vaccine strains was often observed.

Vaccination of birds with the Megan® Vac1 strain, that carries deletions in the cya and crp genes (U.S. Pat. No. 5,389,368; U.S. Pat. No. 5,855,879; U.S. Pat. No. 5,855,880) results in a reduction of the number of birds from which a virulent Salmonella challenge strain can be isolated 7 days after challenge. However, it does not provide a full protection (http://www.meganhealth.com/meganvac.html).

There is thus still a need for improved live attenuated Salmonella vaccine strains, and for improved live attenuated vaccine strains of bacteria infecting veterinary species in general.

Information in literature on the influence of guaB mutations on the virulence of Salmonella and other bacteria is sparse.

McFarland and Stocker (1987, Microbial pathogenesis 3:129-141) reported on the reduced virulence of guaA and guaB Tn10 insertion mutants of S. Typhimurium and S. dublin in BALB/c mice. At high dosage (2.5×10⁷ cfu for S. Typhimurium and 10⁴ cfu for S. dublin), however, these authors reported a high lethality, resulting from the multiplication of the auxotrophic strain.

Wang et al. (2001, Infection and Immunity 69:4734-4741) reported on pre-clinical trials with a vaccine against typhoid fever in humans. The vaccine of Wang et al. comprised a ΔguaBA mutant of Salmonella typhi. This attenuated strain, however, showed a significant residual virulence in mice.

International patent application WO 99/58146 and U.S. Pat. No. 6,190,669 disclose Salmonella typhi vaccine strains harboring a ΔguaBA mutation, which are used as a live vector to deliver foreign antigens.

### Aims of the Invention

An object of the present invention is to provide attenuated Salmonella enterica strains.

Another object of the present invention is to provide a live attenuated vaccine against Salmonellosis and methods of prevention based thereon.

Yet another object of the present invention is to provide live attenuated strains which are useful as live vector and as DNA-mediated vaccines expressing foreign antigens. Such strains are highly suitable for the development of vaccines including polyvalent vaccines.

Still another object of this invention is to provide a method to achieve a S. enterica deletion mutant for use in a live attenuated vaccine.

Yet a further object of this invention is to provide the same materials and methods for the preparation of attenuated strains of bacteria infecting veterinary specie, poultry more in particular.

The general aim is to improve food safety and animal health.

### SUMMARY OF THE INVENTION

A first aspect of the invention relates to an attenuated Salmonella enterica mutant strain which is incapable of forming de novo guanine nucleotides, and wherein said mutant contains a deletion mutation in the guaB gene.

The principle as demonstrated here for S. enterica can be applied to any organism that can use the guanine nucleotide as an intermediary. An aspect of the invention therefore relates to an attenuated mutant strain of a bacterium infecting veterinary species that contains a deletion mutation in the guaB gene. The term “bacterium infecting veterinary species” in the context of the invention refers in particular to bacteria that are pathogenic to veterinary species, and which can be attenuated by a deletion mutation in the guaB gene. The bacterium infecting veterinary species may be a Gram-negative bacterium. Preferred are Gram-negative bacteria for poultry such as Salmonella, Pasteurella, Escherichia coli, etc. Most preferred are Salmonella enterica and (pathogenic) E. coli. By “pathogenic to” is meant that the bacterium, if not attenuated, is capable of causing an infectious disease in the veterinary species.

The mutants of the invention fail to express a functional GuaB gene product. In other words the guaB gene function is impaired, whereby an auxotrophic attenuated strain is obtained.

The mutant strain of the invention, carrying a deletion mutation in the guaB gene, can not grow on Minimal A medium, unless this medium is supplemented with 0.3 mM guanine, xanthine, guanosine or xanthosine.

The present invention in particular aims to provide attenuated S. Enteritidis and S. Typhimurium strains.

Preferably the deletion mutation in the guaB gene is introduced into parent strain S. Enteritidis phage type 4 strain 76Sa88 or into parent strain S. Typhimurium 1491S96.

One of the attenuated S. enterica strains obtained according to the invention is a S. Enteritidis strain SM69 having the deposit number LMG P-21641. Another example is the S. Typhimurium strain SM86 having the deposit number LMG P-21646.

The attenuated mutant strains of the invention are highly suitable for use in a live attenuated vaccine including a polyvalent or multivalent vaccine. The attenuated mutant strain of the invention may encode and express a foreign antigen and may as such be used as a live vector and/or as a DNA-mediated vaccine.

A second aspect of the invention concerns a pharmaceutical composition or a vaccine for immunizing a veterinary species against a bacterial infection (e.g. Salmonellosis caused by Salmonellae) comprising:

a pharmaceutically effective or an immunizing amount of a mutant strain according to the invention, which is incapable of forming de novo guanine nucleotides due to a deletion mutation in the guaB gene; and

a pharmaceutically acceptable carrier or diluent. Preferred compositions are those comprising a Salmonella enterica mutant strain according to the invention.

The attenuated strain of the invention may transfer DNA encoding a foreign antigen in a eukaryotic cell. This foreign antigen may be encoded by and expressed from a plasmid comprised by the attenuated mutant strain of the invention.

In general about 10² cfu to about 10¹⁰ cfu, preferably about 10⁵ cfu to about 10¹⁰ cfu is administered (examples of a pharmaceutically effective or an immunizing amount). An immunizing dose varies according to the route of administration. Those skilled in the art may find that the effective dose for a vaccine administered parenterally may be smaller than a similar vaccine which is administered via drinking water, and the like.

The attenuated strains of the invention, and pharmaceutical compositions or vaccines comprising same, are highly suitable for immunizing an animal or veterinary species against a bacterial infection (e.g. Salmonellosis) and possibly other diseases (in the case of a multivalent vaccine).

A further aspect of the invention therefore concerns a method of immunizing animals, preferably veterinary species, more preferably poultry such as chicken against an infection by a bacterium (e.g. Salmonellosis caused by Salmonellae), said method comprising the step of:

administering to the animal or veterinary species in need thereof an immunizing amount of an attenuated mutant strain of the invention and/or of a vaccine comprising same, whereby a protective immune response is then invoked in the animal or veterinary species.

Examples of veterinary species to be immunized against Salmonellosis: poultry, small or heavy livestock such as chicken, turkey, ducks, quails, guinea fowl, pigs, sheep, young calves, cattle etc.

In general an appropriate dose is administered to these animals, preferably via the oral, nasal or parenteral route.

A last aspect of the invention relates to a mutant strain of the invention for use as a medicament (e.g. for use in a vaccine). Yet another aspect of the invention relates to the use of an attenuated mutant strain of the invention for the preparation of a medicament, such as a vaccine, for the prevention (and/or treatment) of a disease caused by a pathogen (the infecting bacterium) such as Salmonellosis. Examples of animals or veterinary species to be treated and recommended doses are given above.

### DETAILED DESCRIPTION OF THE INVENTION

It was surprisingly found that a deletion mutation in the guaB gene can lead to attenuated Salmonella enterica strains with significantly reduced virulence and capable of inducing an immune response in a livestock animal. Such deletion would attenuate any organism that can use the guanine nucleotide as an intermediary.

The term “gene” as used herein refers to the coding sequence and its regulatory sequences such as promoter and termination signals.

The deletion mutant according to the invention is one in which the purine metabolic pathway enzyme IMP dehydrogenase (encoded by guaB) is inactivated.

Such inactivation may be obtained via a deletion by which the guaB gene function is impaired, leading to a null-function (no functional gene product formed) of the affected gene(s). A person skilled in the art knows how to obtain such mutants and a simple test can tell whether the guaB gene function is impaired. The mutant strain which fails to express a functional guaB gene product cannot grow on Minimal A medium, unless this medium is supplemented with (e.g. 0.3 mM) guanine, xanthine, guanosine or xanthosine.

The invention aims to provide, amongst others, attenuated S. Enteritidis and S. Typhimurium strains since these are the most common S. enterica serovars.

The present invention provides attenuated strains. The invention provides amongst others attenuated Salmonella enterica strains for use, inter alia, as live attenuated vaccines against Salmonellosis, as live vector and/or as DNA-mediated vaccines expressing foreign antigens. As used herein, a “foreign antigen” means an antigen foreign to Salmonella.

Live vector vaccines, also called “carrier vaccines” and “live antigen delivery systems”, comprise an exciting and versatile area of vaccinology (Levine et al, 1990, Microecol. Ther. 19:23-32). In this approach, a live viral or bacterial vaccine is modified so that it expresses protective foreign antigens of another microorganism, and delivers those antigens to the immune system, thereby stimulating a protective immune response. Live bacterial vectors that are being promulgated include, among others, attenuated Salmonella.

An object of the invention is to provide attenuated strains, like attenuated S. enterica strains for use in a live vaccine, possibly a polyvalent or multivalent live vaccine.

One of the objects of the invention is therefore to provide a vaccine against e.g. Salmonellosis comprising:

a pharmaceutically effective or an immunizing amount of a mutant of the invention (e.g. a Salmonella enterica mutant) which is incapable of forming de novo guanine nucleotides, wherein said mutant contains a deletion mutation in the guaB gene; and

a pharmaceutically acceptable carrier or diluent.

Another object of the invention is to provide a live vector vaccine comprising:

a pharmaceutically effective or an immunizing amount of a mutant of the invention (e.g. a Salmonella enterica mutant), which is incapable of forming de novo guanine nucleotides, wherein said mutant contains a mutation in the guaB gene, and wherein said mutant encodes and expresses a foreign antigen; and

a pharmaceutically acceptable carrier or diluent.

The particular foreign antigen employed in the (S. enterica) live vector is not critical to the present invention.

Still another object of the invention is to provide a DNA-mediated vaccine comprising:

a pharmaceutically effective amount or an immunizing amount of a mutant of the invention (e.g. a Salmonella enterica mutant), which is incapable of forming de novo guanine nucleotides, wherein said mutant contains a mutation in the guaB gene; wherein said mutant contains a plasmid which encodes and expresses in a eukaryotic cell, a foreign antigen; and

a pharmaceutically acceptable carrier or diluent.

Details as to the construction and use of DNA-mediated vaccines can be found in U.S. Pat. No. 5,877,159, which is incorporated by reference herein in its entirety. Again, the particular foreign antigen employed in the DNA-mediated vaccine is not critical to the present invention.

The decision whether to express the foreign antigen in e.g. S. enterica (using a prokaryotic promoter in a live vector vaccine) or in the cells invaded by e.g. S. enterica (using a eukaryotic promoter in a DNA-mediated vaccine) may be based upon which vaccine construction for that particular antigen gives the best immune response in animal studies or in clinical trials, and/or, if the glycosylation of an antigen is essential for its protective immunogenicity, and/or, if the correct tertiary conformation of an antigen is achieved better with one form of expression than the other (U.S. Pat. No. 5,783,196).

In the vaccines of the present invention, the pharmaceutically effective amount or the immunizing amount of the mutants of the present invention to be administered will vary depending on the age, weight and sex of the subject. By an “immunizing amount” as used herein is in fact meant an amount that is able to induce an immune response in the animal that receives the pharmaceutical composition/vaccine. The immune response invoked may be a humoral, mucosal, local and/or a cellular immune response.

The particular pharmaceutically acceptable carrier or diluent employed is not critical to the present invention, and are conventional in the art. Examples of diluents include: buffer for buffering against gastric acid in the stomach, such as citrate buffer (pH 7.0) containing sucrose, bicarbonate buffer (pH 7.0) alone, or bicarbonate buffer (pH 7.0) containing ascorbic acid, lactose, and optionally aspartame. Examples of carriers include: proteins, e.g., as found in skimmed milk; sugars; e.g. sucrose; or polyvinylpyrrolidone.

The deletion mutants according to the invention have been created via standard homologous recombination techniques, whereby part of the guaB gene, for instance part of the guaB coding sequence, in a first step is replaced by a resistance gene and flanking FRT sites.

Preferably, in a second step, said resistance gene is removed by recombination between the two FRT sites. One FRT site and the priming sites P1 and P2 remain by the molecular mechanism of the recombination removing the antibiotics resistance gene according to Datsenko and Wanner (2000) (see for instance FIG. 4).

A particular example of the invention relates for instance to guaB deletion mutants of S. Enteritidis that comprise a mutated guaB gene or coding sequence comprising SEQ ID NO: 12.

### SHORT DESCRIPTION OF THE DRAWINGS

FIG. 1 is a schematic representation of the biosynthetic pathway of guanosine monophosphate (adapted from Zalkin and Nygaard, 1996, in “Escherichia coli and Salmonella, Cellular and Molecular Biology, Second edition”, 1996 F. C. Neidhardt ed. ASM Press, Washington D.C., Vol. 1, Ch. 34:561-579). AICAR: 5′-phosphoribosyl-4-carboxamide-5-aminoimidazole; ATP: adenosine triphosphate; G: guanine; GMP: guanosine monophosphate; GR: guanosine; Hx: hypoxanthine; HxR: hypoxanthine riboside (inosine); IMP: Inosine monophosphate; X: Xanthine, XMP: Xanthosine monophosphate; guaA: GMP synthetase, guaB: IMP dehydrogenase; guaC: GMP reductase.

FIG. 2 represents contig 1294 of the S. Enteritidis genome (SEQ ID NO: 10). The ATG initiation codon and TGA termination codon of the guaB gene are in bold.

FIG. 3 represents the sequence of the ΔguaB fragment of S. Enteritidis cloned in pUC18 (SEQ ID NO: 11). The primers that were used are indicated by horizontal arrows. The fragment generated with primers GuaB6-GuaB7 was cloned in pUC18. The ATG initiation and TGA termination codon of the guaB gene and the CCCGGG SmaI restriction site are indicated in bold.

FIG. 4 represents the nucleotide sequence of the S. Enteritidis PCR fragment, which includes the guaB deletion, obtained after sequencing, using primer GuaB10 (SEQ ID NO: 12). The PCR fragment was amplified with primers GuaB6-GuaB7, using total genomic DNA of the mutant SM20. The remaining FRT site is indicated in bold italic and the P1 and P2 primers by arrows (Datsenko and Wanner, 2000, PNAS 97:6640-6645). The ATG initiation and TGA termination codon of the guaB gene are indicated in bold.

FIG. 5 represents the guaB gene of S. Typhimurium LT2, section 117 of 220 of the complete genome (SEQ ID NO: 13). The ATG initiation codon and TGA termination codon of the guaB gene are in bold.

FIGS. 6-7 represent the deposit receipts of SM69 and SM86 respectively.

The invention will be described in further details in the following examples and embodiments by reference to the enclosed drawings. Particular embodiments and examples are not in any way intended to limit the scope of the invention as claimed.

### EXAMPLES

### Example 1

### Auxotrophic Mutation Affects the guaB Gene

An auxotrophic insertion mutant of a wild type S. Enteritidis was obtained via insertion mutagenesis. Only when supplemented with 0.3 mM guanine, xanthine, guanosine or xanthosine could the mutant strain grow on Minimal A medium.

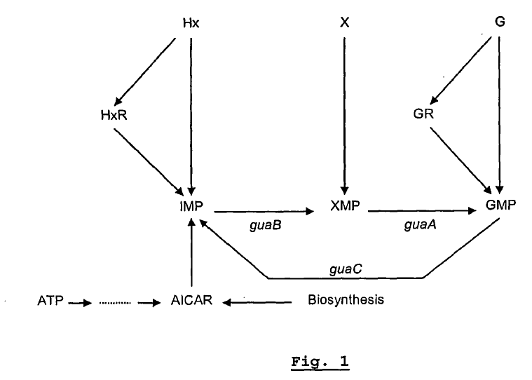

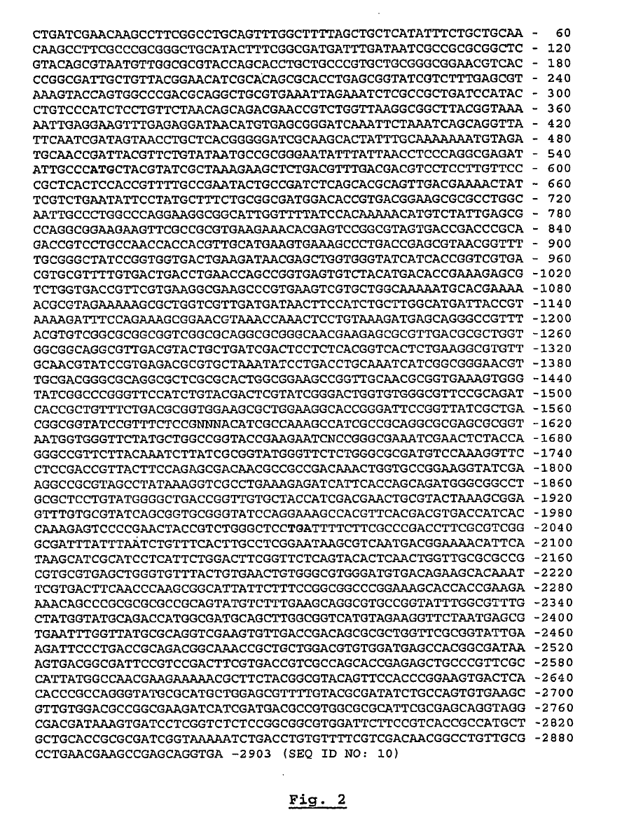

These data strongly suggest that the auxotrophic mutation of the strain affects the guaB gene, encoding the enzyme IMP dehydrogenase (EC 1.1.1.205). This enzyme converts inosine-5′-monophosphate (IMP) into xanthosine monophosphate (XMP) as indicated in FIG. 1.

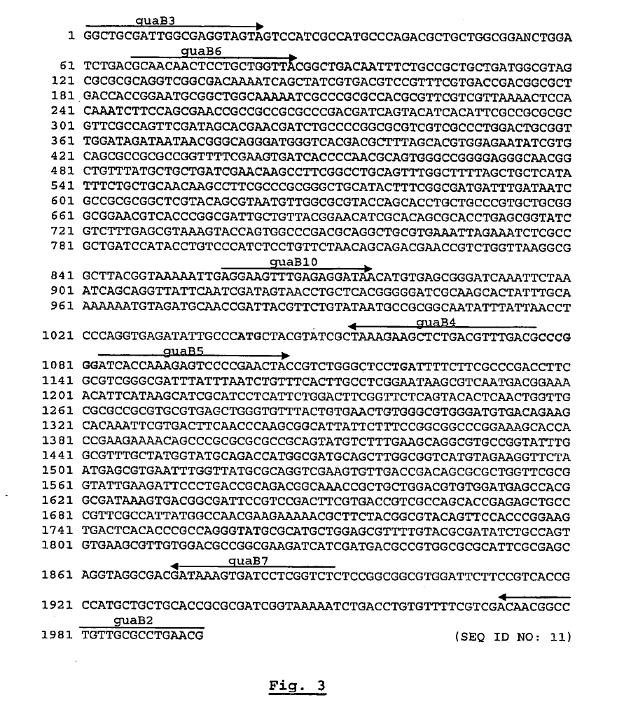

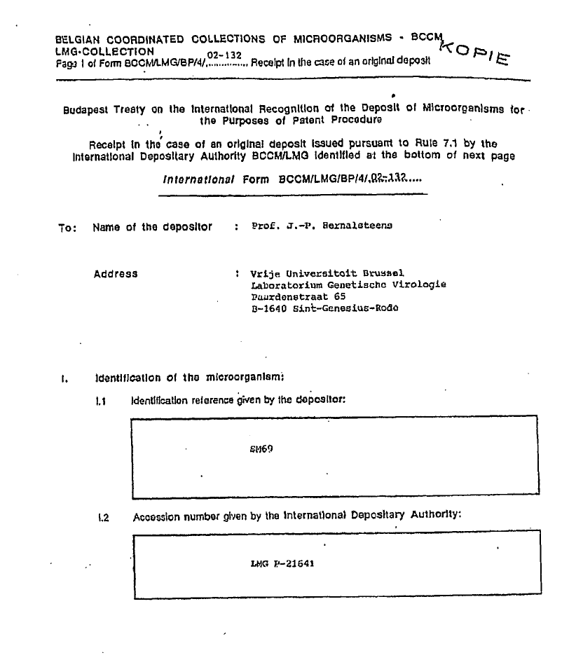

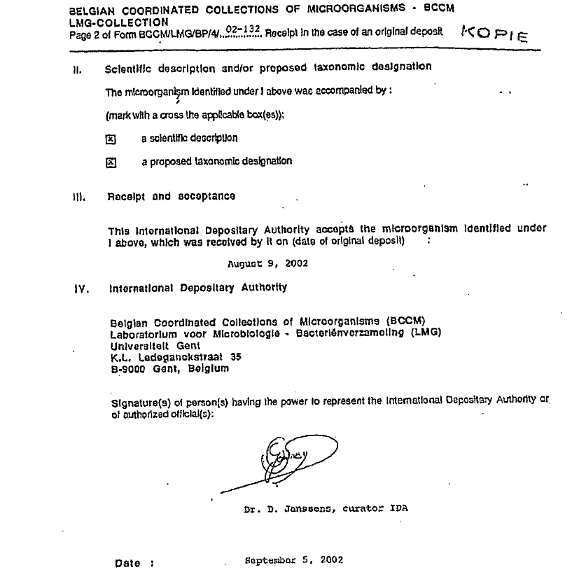

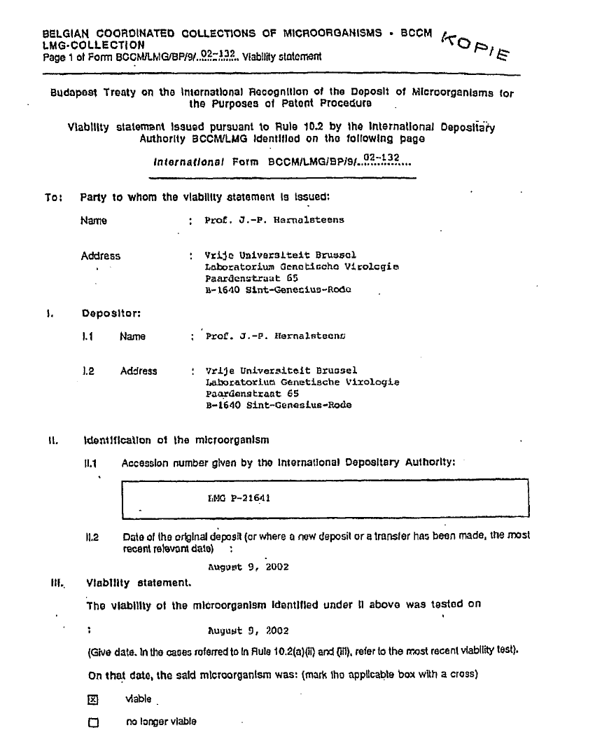

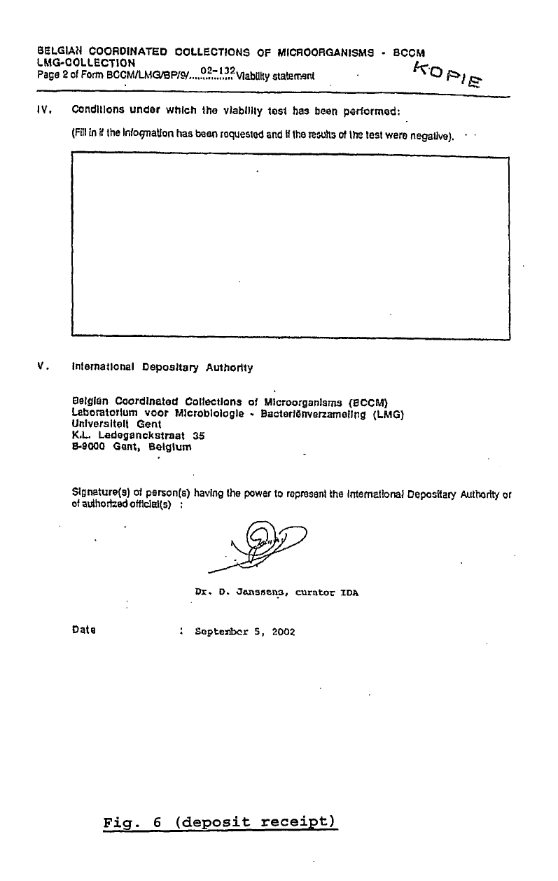

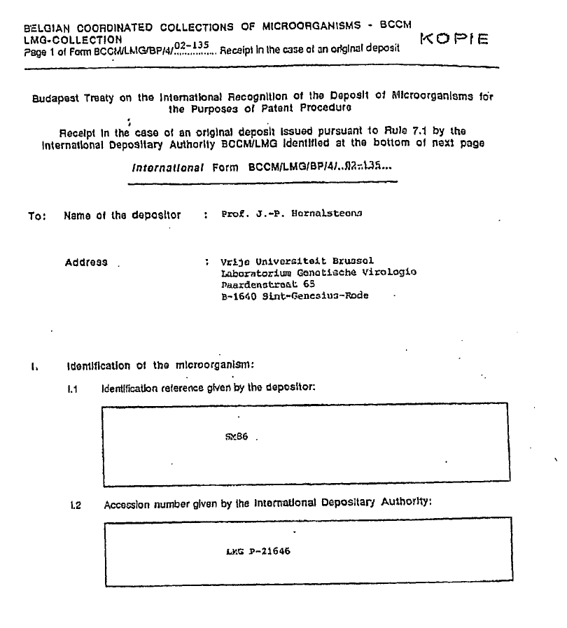

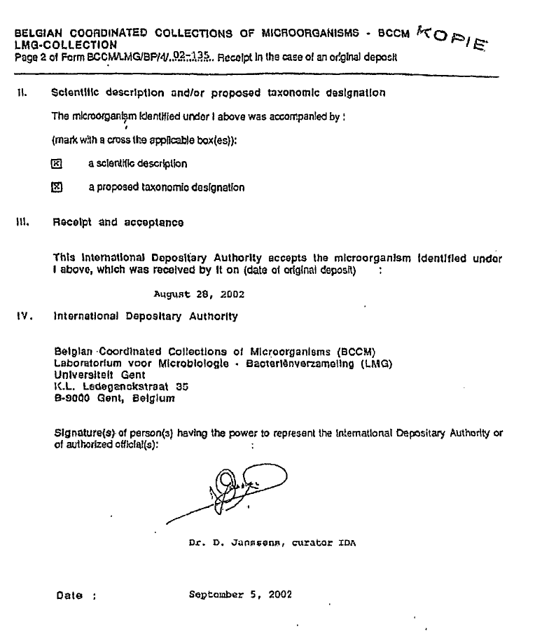

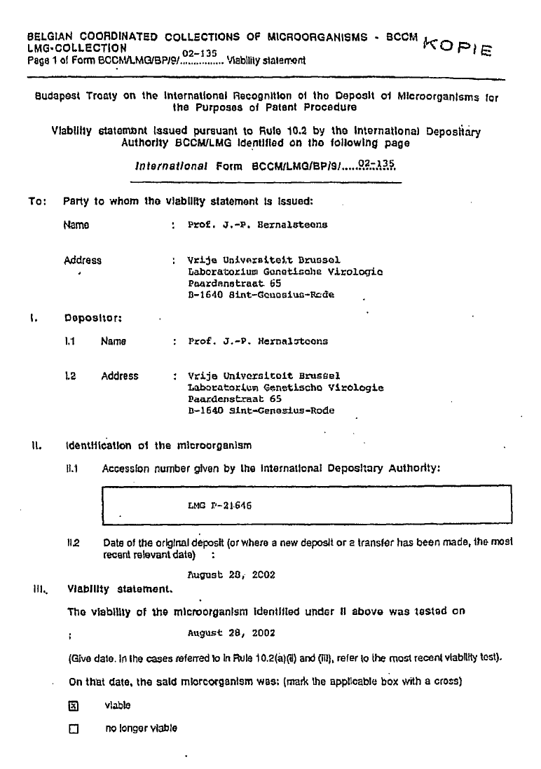

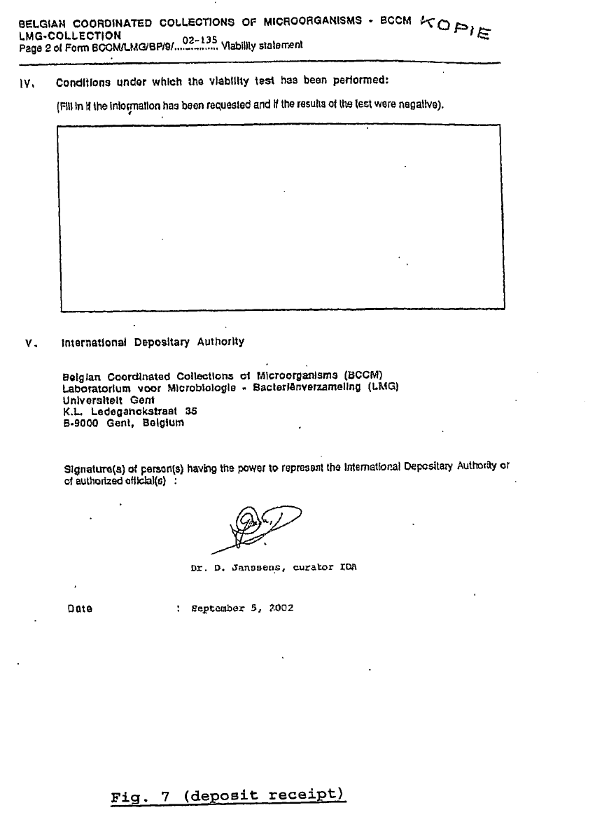

An insertion mutant can revert, thereby restoring the pathogenicity of the strain. This limits its applicability in a live attenuated vaccine. In that aspect deletion mutants are preferred. guaB deletion mutants of S. Enteritidis and S. Typhimurium were therefore created and tested. The guaB genes of both serovars are given in FIGS. 2 and 5.

### Example 2

### guaB Deletion Mutants

Construction of guaB Deletion Mutants

guaB deletion mutants were created according to the method for generating deletion mutations in the genome of Escherichia coli K12 (Datsenko and Wanner, 2000, PNAS 97:6640-5, incorporated by reference herein).

This method relies on homologous recombination, mediated by the bacteriophage λ Red recombinase system, of a linear DNA fragment generated by PCR.

The guaB sequence is hereby substituted by an antibiotic resistance gene. This resistance gene is flanked by FRT sites (FLP recognition target sites) and can be excised from the genome by site-specific recombination, mediated by the FLP recombinase.

Overlap PCR (Ho et al., 1989, Gene 77:51-59) was applied to construct a linear fragment. The principle relies on the use of two primer sets, one upstream pair (GuaB3-GuaB4; GuaB3: 5′ GGCTGCGATT GGCGAGGTAG TA 3′, SEQ ID NO 2; GuaB4: 5′ GGTGATCCCG GGCGTCAAAC GTCAGGGCTT CTTTA 3′, SEQ ID NO 3) and one downstream pair (GuaB5-GuaB2; GuaB5: 5′ TTGACGCCCG GGATCACCAA AGAGTCCCCG AACTA 3′, SEQ ID NO 4; GuaB2: 5′ CGTTCAGGCG CAACAGGCCG TTGT 3′, SEQ ID NO 1) of the guaB gene.

Both sets contain primers (GuaB4, GuaB5) that are partially complementary and to which a SmaI restriction site was added.

After annealing of the resulting complementary sequences and chain elongation, PCR with the outward primers (GuaB6-GuaB7; GuaBG: 5′ GCAACAACTC CTGCTGGTTA 3′, SEQ ID NO 5; GuaB7: 5′ AGACCGAGGA TCACTTTATC 3′, SEQ ID NO 6) generated a fragment with a 6 basepair SmaI site replacing an 861 basepair internal segment of the guaB coding sequence. This ΔguaB fragment was cloned in the vector pUC18 (see FIG. 3).

The chloramphenicol resistance gene (cat) with its flanking FRT sequences was amplified using the primers P1 (5′ GTGTAGGCTG GAGCTGCTTC 3′, SEQ ID NO 8) and P2 (5′ CATATGAATA TCCTCCTTAG 3′, SEQ ID NO 9) (Datsenko and Wanner, 2000) and plasmid pKD3 DNA (Datsenko and Wanner, 2000) as a template.

This PCR fragment was ligated in the SmaI site of the cloned ΔguaB fragment. The desired fragment was generated using nested primers (GuaB6-GuaB7).

The resulting PCR fragment was electroporated into S. Enteritidis phage type 4 strain 76Sa88 (a clinical isolate from a turkey, obtained from the Veterinary and Agrochemical Research Centre, Groeselenberg 99, B-1180 Ukkel, Belgium) harboring the temperature sensitive replication plasmid pKD46, encoding the bacteriophage Lambda Red recombinase system.

The chloramphenicol resistant transformants were tested on Minimal A medium and on Minimal A medium supplemented with 0.3 mM guanine. The ΔguaB::catFRT mutants were confirmed by. PCR using the following primer combinations: GuaB6-GuaB7, GuaB6-P2, GuaB7-P1 and P1-P2.

The S. Enteritidis ΔguaB::catFRT mutant (SM12) was transformed with the temperature sensitive replication plasmid pCP20 by electroporation. The plasmid pCP20 encodes the FLP recombinase, which recognizes the FRT-sites, to remove the cat gene. The resulting strain was named SM20.

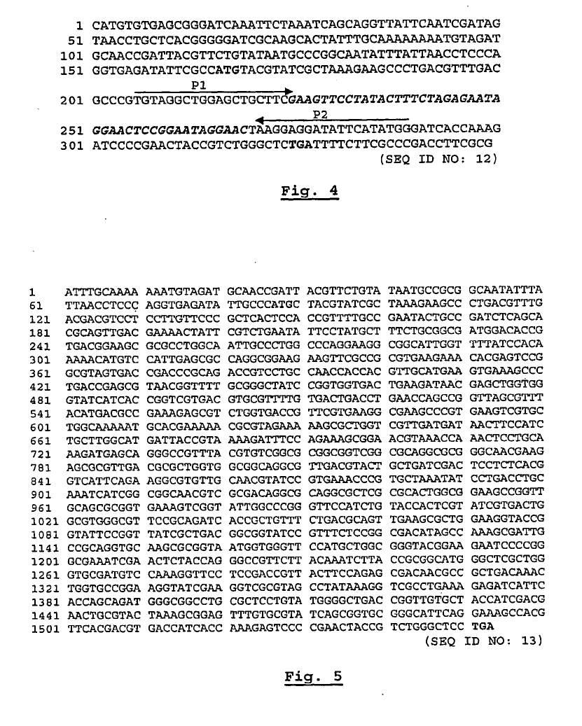

The PCR fragment in which the deletion is located was obtained using total genomic DNA of the mutant SM20 and the primer combination GuaB6-GuaB7 (see FIG. 4).

The ΔguaB mutation was confirmed by sequencing of this fragment, using the primer GuaB10 (5′ AGGAAGTTTG AGAGGATAA 3′, SEQ ID NO 7).

The sequences of all above-mentioned primers are given in Table 1.

To avoid the presence of possible additional mutations, caused by the expression of the Red recombinase system, an isogenic strain was constructed.

The ΔguaB::catFRT mutation of the mutant SM12 was transduced by bacteriophage P22 HT int⁻ (Davis, R. W., Botstein D. and Roth, J. R. (1980) In Advanced Bacterial Genetics, A manual for genetic engineering. Cold Spring Harbor Laboratory, Cold Spring Harbor, N.Y.) to wild type S. Enteritidis 76Sa88. The cat gene was removed using the plasmid pCP20. The resulting strain was called SM69 (deposit number LMG P-21641).

A ΔguaB mutant of S. Typhimurium strain 1491S96 was constructed using the same procedure and the same primers. The resulting strains S. Typhimurium ΔguaB::catFRT and S. Typhimurium ΔguaB were named SM9 and SM19 respectively. SM86 (having the deposit number LMG P-21646) is the isogenic strain obtained therefrom, after transduction by bacteriophage P22 HT int⁻ lysate from strain SM9 and excision of the cat gene using the plasmid pCP20.

The ΔguaB mutants SM19, SM20, SM86 and SM69 are sensitive to bacteriophage P22. This proves the presence of intact lipopolysaccharides (LPS).

Virulence and Protection Tests with the guaB Deletion Mutant SM20 in Mice

The virulence of the mutant SM20 in mice was tested by oral infection of 6-8 week old female BALB/c mice in two independent experiments. These were performed as described above. The wild type strain S. Enteritidis 76Sa88 was tested in parallel as a positive control. The S. Enteritidis 76Sa88 ΔaroA mutant SM50 was included in the experiment as a vaccine control. This mutant carries a precise deletion of the complete aroA coding sequence and was constructed by the method of Datsenko and Wanner (2000).

The complete data are given in Tables 2 and 3. These results demonstrate that the ΔguaB mutant SM20 is strongly attenuated in mice but still shows some residual pathogenicity when administered at this high dose. Oral immunization with the mutant induces protective immunity against infection by a high dose of the corresponding pathogenic wild type S. Enteritidis strain 76Sa88. The protection is at least equal to the protection conferred by the S. Enteritidis ΔaroA mutant SM50.

Virulence and Protection Tests with the Isogenic guaB Deletion Mutants SM69 and SM86 in Mice

The virulence of the mutants SM69 and SM86 in mice was tested by oral infection of 6-8 week old female BALB/c mice. These were performed as described above. The wild type strains S. Enteritidis 76Sa88 and S. Typhimurium 1491S96 were tested in parallel as positive controls.

The complete data are given in Tables 4-7. These results demonstrate that the ΔguaB mutants SM69 and SM86 are strongly attenuated in mice but still show some residual pathogenicity when administered at this high dose. Oral immunization with the mutants induces protective immunity against infection by a high dose of the corresponding pathogenic wild type strain.

Safety Evaluation of the S. Enteritidis guaB Deletion Mutant SM69 in One-Day-Old Chickens by the Intratracheal and Oral Gavage Routes

The objective of this study was to evaluate the safety of S. Enteritidis ΔguaB mutant strain SM69 master seed in one-day-old chickens. Mortality was used as a primary parameter for the determination of safety.

Chicks at one day of age were leg-banded and randomly placed in each of the four treatment groups (Group 1: SM69-IT, group 2: SM69-OG, group 3: PBS-IT and Group 4: PBS-OG). After the master seed inoculation, the birds from groups 1 and 2 were placed in one isolator and those of groups 3 and 4 in another isolator.

Chickens in groups 1 and 2 were inoculated with the SM69 master seed by the intratracheal (IT) route or oral gavage (OG) route, respectively, with an actual titer of 1.28×10⁸ cfu/0.2 ml per bird. Chickens in groups 3 and 4 were administered with 0.2 ml PBS (phosphate buffered saline) per bird by the intratracheal or oral gavage, respectively.

Following the inoculation of SM69 or PBS, chick mortality was observed daily until 38 days post inoculation. Table 8 summarizes the results of mortality for all 4 groups. In group 1, one bird died during the inoculation due to inoculation trauma. Two birds died at 2 days post inoculation (DPI). Three birds died from day 3 to day 13 (at 3, 5 and 13 DPI respectively). A total of 6 birds thus died in group 1. In group 2, two birds died in total. One died during inoculation due to inoculation trauma and one died at day 5 post inoculation. No birds died in the PBS treated groups either by the intratracheal or oral gavage route.

This study indicates that the S. Enteritidis ΔguaB mutant strain SM69 is not safe when administered at 1.28×10⁸ cfu per bird at one day of age by the intratracheal or oral gavage route.

Safety Evaluation of the S. Enteritidis ΔguaB Deletion Mutant SM69 in 2-Week Old Chickens by the Intratracheal and Oral Gavage Routes

Safety of the S. Enteritidis ΔguaB mutant strain SM69 was then evaluated in 2 week-old specific pathogen free (SPF) chickens by the intratracheal and oral gavage routes. Mortality was used as a primary criterion and body weight as a secondary criterion for the determination of safety.

Birds at 2 weeks of age were leg-banded and randomly placed in each of the four treatment groups: SM69-IT, SM69-OG, Poulvac ST-IT and PBS-IT. Ten birds in group 1 were inoculated with SM69 by the intratracheal route; ten birds in group 2 were inoculated with SM69 by oral gavage; ten birds in group 3 were inoculated with a S. Typhimurium AroA⁻ vaccine (Poulvac® ST) by the intratracheal route; and five birds in group 4 were inoculated with PBS by the intratracheal route.

Chickens in groups 1 and 2 were inoculated with SM69 master seed by the intratracheal or oral gavage route, respectively, with the actual titer of 2.304×10⁸ cfu/0.2 ml per bird. Chickens in group 3 were administered with Poulvac® ST by the intratracheal route with 2.19×10⁸ cfu/0.2 ml per bird. Chickens in group 4 were administered by the intratracheal route with 0.2 ml PBS per bird.

After inoculation, the birds from treatment groups 1 and 2 were placed in one isolator and those from groups 3 and 4 in another isolator.

Following inoculations, mortality was observed daily until 21 days post-inoculation. Body weight of all birds was also recorded at the end of the study period (21 days). Poulvac® ST and PBS were used as intratracheal procedure controls.

During the 21-day observation period, one bird in the SM69 intratracheal treatment group (group 1) died from an infected yolk sac. No mortality was associated with SM69 inoculation, indicating that the SM69 strain is safe at the titer tested, 2.304×10⁸ cfu per bird by the intratracheal and oral gavage routes. As expected, no death was observed either in the Poulvac® ST treated birds at the titer of 2.19×10⁸ cfu per bird or in the PBS treated birds, indicating that the study was valid (Table 9).

Body weight was compared amongst groups in an analysis of variance (ANOVA) model with body weight as the dependent variable and treatment included as an independent variable. Group comparisons were made using Tukey's test for multiple comparisons. The level of significance was set a p<0.05. The study was considered valid because the control chickens (PBS control group) remained healthy and free of clinical signs of diseases or mortality throughout the study.

There were no significant differences in the final body weight in chickens administered with SM69 by the intratracheal or oral gavage inoculation, Poulvac® ST, or PBS (Table 5). Even though no baseline was established of the birds in each group at one day of age, it was unlikely that there was a significant difference in the initial body weight amongst the four groups since the birds were randomly placed into each of the 4 treatment groups.

Since no mortality was attributable to the inoculation of SM69, it can be concluded that the S. Enteritidis ΔguaB mutant strain, SM69, is safe when administered at the tested titer of 2.304×10⁸ cfu/0.2 ml dose per 2-week-old bird, by either the intratracheal or oral gavage inoculation route. SM69 inoculations had no effect on the final body weight of the birds, bird weight being the second safety parameter evaluated here.

A deposit has been made according to the Budapest Treaty at the BCCM/LMG Culture Collection, Laboratorium voor Microbiologie, K.L. Ledeganckstraat 35, B-9000 Gent (Belgium) for the following micro-organisms: Salmonella Enteritidis SM69 under deposit number LMG P-21641 (deposit date: 9 Aug., 2002) and S. Typhimurium SM86 under deposit number LMG P-21646 (deposit date: 28 Aug., 2002). The deposits have been made in the name of Prof. J. -P. Hernalsteens, previous address: Vrije Universiteit Brussel, Laboratorium Genetische Virologie, Paardenstraat 65, B-1640 Sint-Genesius-Rhode, current address: Vrije Universiteit Brussel, Onderzoeksgroep Genetische Virologie, Pleinlaan 2, B-1050 Brussels, Belgium.

TABLE 1
Primer sequences

| SEQ | | |
|---|---|---|
| ID | | |
| NO | Primer | Sequence |
| 1 | GuaB2 | 5′ CGTTCAGGCGCAACAGGCCGTTGT 3′ |
| 2 | GuaB3 | 5′ GGCTGCGATTGGCGAGGTAGTA 3′ |
| 3 | GuaB4 | 5′ GGTGATCCCGGGCGTCAAACGTCAGGGCTTCTTTA 3′ |
| 4 | GuaB5 | 5′ TTGACGCCCGGGATCACCAAAGAGTCCCCGAACTA 3′ |
| 5 | GuaB6 | 5′ GCAACAACTCCTGCTGGTTA 3′ |
| 6 | GuaB7 | 5′ AGACCGAGGATCACTTTATC 3′ |
| 7 | GuaB10 | 5′ AGGAAGTTTGAGAGGATAA 3′ |
| 8 | P1 | 5′ GTGTAGGCTGGAGCTGCTTC 3′ |
| 9 | P2 | 5′ CATATGAATATCCTCCTTAG 3′ |

TABLE 2
Virulence test in BALB/c mice of the S. Enteritidis guaB deletion mutant SM20

| Infection | | | Day of | |
|---|---|---|---|---|
| Strain | Dose | Survival | death | State of the mice |

First Experiment

| | | | | |
|---|---|---|---|---|
| negative control: milk | / | 11/11 | / | No disease symptoms |
| positive control: S. Enteritidis 76Sa88 | 2.1 × 10⁸ | 0/5 | 7, 7, 8, | |
| | | | 8, 9 | |
| vaccine control: S. Enteritidis ΔaroA SM50 | 2.5 × 10⁸ | 10/10 | / | No disease symptoms |
| S. Enteritidis ΔguaB SM20 | 5.1 × 1O⁸ | 9/10 | 13 | Disease symptoms between |
| | | | | the 7^{th} and the 14^{th} day |
| | | | | after infection |

Second Experiment

| | | | | |
|---|---|---|---|---|
| negative control: milk | / | 4/4 | / | No disease symptoms |
| positive control: S. Enteritidis 76Sa88 | 1.4 × 10⁸ | 0/3 | 8, 9, 9 | |
| vaccine control: S. Enteritidis ΔaroA SM50 | 2.1 × 10⁸ | 3/3 | / | No disease symptoms |
| S. Enteritidis ΔguaB SM20 | 1.9 × 10⁸ | 3/3 | / | No disease symptoms |

TABLE 3
Challenge of mice vaccinated with the S. Enteritidis guaB deletion mutant SM20

| Vaccination | | Challenge | | | Day of | |
|---|---|---|---|---|---|---|
| Strain | Dose | Strain | Dose | Survival | death | State of the mice |

First Experiment

| | | | | | | |
|---|---|---|---|---|---|---|
| negative | / | negative | / | 6/6 | / | No disease symptoms |
| control: milk | | control: milk | | | | |
| negative | / | S. Enteritidis | 1.5 × 10⁸ | 0/5 | 7, 8, 8, | Disease symptoms starting |
| control: milk | | 76Sa88 | | | 8, 9 | on the 5^{th} day after |
| | | | | | | challenge |
| Vaccine | 2.5 × 10⁸ | S. Enteritidis | 1.5 × 10⁸ | 3/5 | 9, 13 | Disease symptoms between |
| control: | | 76Sa88 | | | | the 7^{th} and the 14^{th} day |
| S. Enteritidis | | | | | | after challenge |
| ΔaroA SM50 | | | | | | |
| S. Enteritidis | 5.1 × 10⁸ | S. Enteritidis | 1.5 × 10⁸ | 5/5 | / | Mice are less active |
| ΔguaB SM20 | | 76Sa88 | | | | between the 11^{th} and the |
| | | | | | | 14^{th} day after challenge. |

Second Experiment

| | | | | | | |
|---|---|---|---|---|---|---|
| negative | / | negative | / | 2/2 | / | No disease symptoms |
| control: milk | | control: milk | | | | |
| negative | / | S. Enteritidis | 1.5 × 10⁸ | 0/2 | 9, 18 | |
| control: milk | | 76Sa88 | | | | |
| Vaccine | 2.1 × 10⁸ | S. Enteritidis | 1.5 × 10⁸ | 1/3 | 9, 21 | Disease symptoms between |
| control: | | 76Sa88 | | | | the 7^{th} and the 21^{st} day |
| S. Enteritidis | | | | | | after challenge. |
| ΔaroA SM50 | | | | | | |
| S. Enteritidis | 1.9 × 10⁸ | S. Enteritidis | 1.5 × 10⁸ | 2/3 | 10 | Disease symptoms starting |
| ΔguaB SM20 | | 76Sa88 | | | | on the 9^{th} day after |
| | | | | | | infection. |

TABLE 4
Virulence test in BALB/c mice with the isogenic S. Enteritidis guaB deletion mutant SM69

| Infection | | | Day of | |
|---|---|---|---|---|
| Strain | Dose | Survival | death | State of the mice |
| negative control: milk | / | 4/4 | / | Asymptomatic |
| positive control: S. Enteritidis 76Sa88 | 3.7 × 10⁸ | 0/3 | 7, 8, 9 | Severe symptoms onwards from |
| | | | | day 5 |
| S. Enteritidis ΔguaB SM69 | 7.6 × 10⁸ | 5/5 | / | Mild symptoms, from day 11 |
| | | | | till day 18 |

TABLE 5
Challenge of mice vaccinated with the S. Enteritidis guaB deletion mutant SM69

| Vaccination | | Challenge | | | Day of | |
|---|---|---|---|---|---|---|
| Strain | Dose | Strain | Dose | Survival | death | State of the mice |
| negative | — | S. Enteritidis | 3.1 × 10⁸ | 0/4 | 8, 8, 8, 9 | Severe symptoms onwards from |
| control: milk | | wild type | | | | day 5 |
| | | strain 76Sa88 | | | | |
| S. Enteritidis | 7.6 × 10⁸ | S. Enteritidis | 3.1 × 10⁸ | 2/5 | 8, 8, 19 | Severe symptoms onwards from |
| ΔguaB SM69 | | wild type | | | | day 5 |
| | | strain 76Sa88 | | | | |

TABLE 6
Virulence experiments with S. Typhimurium 1491S96 isogenic mutant strains in
BALB/c mice. Oral inoculation with of the mutants of S. Typhimurium strain

| Infection | | | | |
|---|---|---|---|---|
| Strain | | | Day of | |
| (S. Typhimurium 1491S96) | Dose | Survival | death | State of the mice |

First Experiment

| | | | | |
|---|---|---|---|---|
| Wild type SM2 | * | 0/3 | (9, 9, 10) | Disease symptoms from day 4 |
| | | | | onwards |
| ΔguaB SM86 | * | 2/5 ** | (2, 2, 2) | Mild disease symptoms from |
| | | | | day 9 until challenge |
| Negative control: milk | — | 4/4 | | No symptoms |

Second Experiment

| | | | | |
|---|---|---|---|---|
| Wild type SM2 | 0.8 × 10⁸ | 1/4 | (11, 13, 14) | Disease symptoms from day 6 |
| | | | | onwards |
| ΔguaB SM86 | 0.8 × 10⁸ | 5/5 | | Weak symptoms on day 13 and 14 |
| Negative control: milk | | 5/5 | | No symptoms |

* inoculated with approximately 10⁸ cells (exact titer not determined)
** died after a fight

TABLE 7
Protection experiments with S. Typhimurium isogenic mutant strains in BALB/c mice.
Oral inoculation with approximately 10⁸ cells of the S. Typhimurium strain 1491S96

| Vaccination | | | | | | |
|---|---|---|---|---|---|---|
| Strain | | | | | | |
| S. Typhimurium | | Challenge | | | Day of | |
| 1491S96 | Dose | Strain | Dose | Survival | death | State of the mice |

First Experiment

| | | | | | |
|---|---|---|---|---|---|
| ΔguaB SM86 | * | S. Typhimurium | 1.3 × 10⁷ | 2/2 | Mild symptoms until days 14, |
| | | 1491S96 | | | afterwards one mouse showed |
| | | | | | clear symptoms, the other |
| | | | | | was healthy again |
| Negative | — | S. Typhimurium | 1.3 × 10⁷ | 0/4 | Severe symptoms from day 6 |
| control: milk | | 1491S96 | | | onwards |

Second Experiment

| | | | | | | |
|---|---|---|---|---|---|---|
| ΔguaB SM86 | 0.8 × 10⁸ | S. Typhimurium | 2.7 × 10⁸ | 5/5 | / | Reduced activity from day 6 |
| | | 1491S96 | | | | till day 16 |
| Negative | — | S. Typhimurium | 2.7 × 10⁸ | 0/5 | (8, 9, 10, | Severe symptoms from day 6 |
| control: milk | | 1491S96 | | | 11, 16) | onwards |

* inoculated with approximately 10⁸ cells (exact titer not determined)

TABLE 8
Safety evaluation of the S. Enteritidis guaB deletion mutant
SM69 in one-day-old chickens

| Infection | | | | | | |
|---|---|---|---|---|---|---|
| Strain | Group | N | Titer | Route | Survival | Day of death (DPI) |
| S. Enteritidis SM69 | 1: SM69-IT | 10 | 1.28 × 10⁸ | intratracheal | 4/10* | 0, 2, 3, 5, 13 |
| | | | cfu/0.2 ml | | | |
| S. Enteritidis SM69 | 2: SM69-OG | 10 | 1.28 × 10⁸ | oral gavage | 8/10** | 0, 5 |
| | | | cfu/0.2 ml | | | |
| negative control: | 3: PBS-IT | 10 | PBS - 0.2 ml | intratracheal | 10/10 | — |
| PBS | | | | | | |
| negative control: | 4: PBS-OG | 10 | PBS - 0.2 ml | oral gavage | 10/10 | — |
| PBS | | | | | | |

IT Intratracheal
OG Oral gavage
DPI Days post inoculation
*In group 1, 1 bird died during inoculation; 1 bird died at 3, 5 and 13 DPI; and 2 birds at 2 DPI respectively
**In group 2, 1 bird died during inoculation; and 1 bird died at 5 days DPI

TABLE 9
Safety evaluation of the S. Enteritidis guaB deletion mutant
SM69 in 2-week-old chickens

| Infection | | | | | | Day of | Mean weight | Std |
|---|---|---|---|---|---|---|---|---|
| Strain | Group | N | Titer | Route | Survival | death (DPI) | (kg) | weight |
| S. Enteritidis SM69 | 1: SM69-IT | 10 | 2.304 × 10⁸ | IT | 9/10* | 13 | 0.429 | 0.064 |
| | | | cfu/0.2 ml | | | | | |
| S. Enteritidis SM69 | 2: SM69-OG | 10 | 2.304 × 10⁸ | OG | 10/10 | — | 0.420 | 0.044 |
| | | | cfu/0.2 ml | | | | | |
| vaccine control: | 3: Poulvac- | 10 | 2.19 × 10⁸ | IT | 10/10 | — | 0.423 | 0.046 |
| Poulvac ® ST** | IT | | cfu/0.2 ml | | | | | |
| negative control: | 4: PBS-IT | 5 | PBS - 0.2 ml | OG | 5/5 | — | 0.388 | 0.019 |
| PBS | | | | | | | | |

IT Intratracheal
OG Oral gavage
DPI Days post inoculation
*Death due to yolk sac infection
**a live S. Typhimurium AroA⁻ vaccine against S. Typhimurium

1. An attenuated mutant strain of bacterium infecting veterinary species, which is incapable of forming de novo guanine nucleotides, and contains a deletion mutation in the guaB gene.

2. The mutant strain of claim 1, wherein the veterinary species is poultry.

3. The mutant strain of claim 1, which is a Salmonella enterica or a (pathogenic) Escherichia coli strain.

4. The mutant strain of claim 1, wherein said mutant strain encodes and expresses a foreign antigen.

5. The mutant strain of claim 1, which is a S. Enteritidis or a S. Typhimurium strain.

6. The mutant strain of claim 1, wherein said mutation is introduced into parent strain S. Enteritidis phage type 4 strain 76Sa88.

7. The mutant strain of claim 6 which is S. Enteritidis strain SM69 having the deposit number LMG P-21641.

8. The mutant strain of claim 1, wherein said mutation is introduced into parent strain S. Typhimurium 1491S96.

9. The mutant strain of claim 8 which is S. Typhimurium strain SM86 having the deposit number LMG P-21646.

10. A vaccine for immunizing a veterinary species against a bacterial infection comprising: a pharmaceutically effective or an immunizing amount of a mutant strain according to claim 1, and a pharmaceutically acceptable carrier or diluent.

11. The vaccine of claim 10, wherein said mutant strain encodes and expresses a foreign antigen.

12. The vaccine of claim 10, wherein said mutant strain comprises a plasmid which encodes and expresses an exogenous gene in a eukaryotic cell.

13. A method of immunizing a veterinary species against a bacterial infection, comprising administering to a veterinary species in need thereof an effective immunizing amount of a mutant strain according to claim 1 and/or a vaccine according to claim 10.

14. The method of claim 13 wherein the veterinary species is poultry.

15. The method of claim 13, wherein the mutant strain and/or the vaccine is administered via the oral, nasal or parenteral route.

16. A pharmaceutical composition comprising the attenuated mutant strain according to claim 1.

17. (canceled)
